(19) United States
(12) Patent Application Publication
(10) Pub. No.: US 2023/0149183 A1
(43) Pub. Date: May 18, 2023

(54) Holder For An Acetabular Cup Implant

(71) Applicant: Embody Orthopaedic Limited, London (GB)

(72) Inventor: Robert Wozencroft, Epsom Surrey (GB)

(21) Appl. No.: 18/094,016

(22) Filed: Jan. 6, 2023

Related U.S. Application Data
(63) Continuation-in-part of application No. PCT/GB2021/051753, filed on Jul. 9, 2021.

(30) Foreign Application Priority Data
Jul. 9, 2020 (GB) 2010525.0

(51) Int. Cl. A61F 2/46

(52) U.S. Cl. CPC A61F 2/4609; A61F 2/4603; A61F 2002/4627

(57) ABSTRACT

There is provided herein a cover for a medical implant, the cover comprising a body portion and a seal, the body portion comprising a distal end and a proximal end, the proximal end comprising a region adapted for connecting to an introducer and the seal comprising at least one tab extending away in a proximal direction from the body portion. Also disclosed herein are systems incorporating such covers, and methods of manufacturing and using the same. There is also provided an introducer adapted for use with the cover.

### PRIORITY CLAIM

The present application is a continuation-in-part application of International Application No. PCT/GB2021/051753 filed Jul. 9, 2021, which designates the United States and claims priority to United Kingdom Patent Application No. 2010525.0 filed Jul. 9, 2020.

### BACKGROUND OF THE INVENTION

### 1 Field of the Invention

This invention generally relates to covers for medical implants and introducers for securing a cover to a medical implant and, more particularly, to covers, holders and introducers for acetabular cup implants.

### 2 Description of the Related Art

The following descriptions and examples are not admitted to be prior art by virtue of their inclusion within this section.

Acetabular cup implants with large diameter bearings, such hip resurfacing arthroplasty (HRA) cups or large diameter total hip replacement (THR) cups, are a challenge to hold during implantation. The surgeon must forcibly impact the cup into a prepared, slightly undersized acetabular socket during fitting, whilst ensuring that the cup is implanted in the correct angle and orientation which is critical to function. Furthermore, it is essential to protect the super finished bearing surface from damage during this impaction process. Most conventional THR cups consist of an outer shell and separate bearing liner which is inserted after the metal shell is implanted. Therefore, the inside of the metal shell is used to provide holding features for an introducer shaft such as a screw thread or bayonet attachment. However, this area is not available on HRA cups or THR cups with pre-fitted liner because the inside surface forms the bearing surface. Furthermore, the outer surface is usually fully embedded in the bone socket. Previous HRA cup designs have attempted to solve this problem in various ways, but these have resulted in compromises to the designs, either by reducing bearing surface, or reducing outer fixation surfaces or by including holding features which caused snagging and aggravation of the soft tissue structures such as Psoas tendon in close proximity to the cup rim.

A new generation of HRA implants and large diameter THR’s utilizing non-metal bearing materials including zirconia toughened alumina ceramic (ZTA) and cross linked ultra-high molecular weight polyethylene (UHMWPE) pose bigger challenges for cup holding during implantation. In the case of ZTA, due to the constraints of the manufacturing process and hardness of the material, it is technically difficult and expensive to add small holding features sometimes present on metal hip cups. In the case of UHMWPE, it is critical for wear characteristics and material strength that full wall thickness is maintained, so holding features that reduce wall section would be undesirable. In addition, some new generation devices are intended for specific rotational alignment of asymmetric features on the cup which also add complication for cup holding and positioning.

### SUMMARY OF THE INVENTION

Covers and introducers for medical implants as well as systems of such and methods of their use are provided. The following description of various embodiments of covers, systems, introducers, and methods is not to be construed in any way as limiting the subject matter of the appended claims.

Embodiments of a cover for a medical implant comprises a body portion and a seal. The body portion comprises a distal end and a proximal end. The proximal end comprises a region adapted for connecting to an introducer and the seal comprises at least one tab extending away in a proximal direction from the body portion.

Embodiments of a method of fixing a cover to a medical implant comprises adding a fluid to a region of the medical implant which will accept the cover, introducing the cover into the region of the medical implant such that the cover displaces the fluid until the cover abuts a surface of the medical implant, and adding additional force to the cover such that a seal of the cover is compressed between a body portion of the cover and the medical implant.

Embodiments of an introducer comprises a drive shaft, a connector hub, and a latching means to secure the connector hub to a cover for a medical implant.

Embodiments of a system comprises a cover for a medical implant and an introducer, wherein the cover is releasably fixable to the introducer.

Embodiments of a system comprises a medical implant and a cover releasably fixable to an inner surface of the medical implant.

### BRIEF DESCRIPTION OF THE FIGURES

Other objects and advantages of the invention will become apparent upon reading the following detailed description and upon reference to the accompanying drawings in which:

FIG. 1 shows the cover body;

FIG. 2 shows the cover body underside;

FIG. 3 is a side view of the cover body with cross sectioned and detailed view;

FIG. 4 is an exploded view showing all parts of the cover (cover body, seal, and snap-ring);

FIG. 5 shows the assembled cover;

FIG. 6 is an alternative view of the assembled cover;

FIG. 7 is a top view of the cover with cross sectioned and detailed views;

FIG. 8 shows an acetabular cup implant;

FIG. 9 shows a cup implant being filled with a liquid;

FIG. 10 shows the cover as it is offered to the cup implant;

FIG. 11 shows the assembled cover and cup implant;

FIG. 12 is a top view of the cover and cup implant with cross sectioned and detailed views;

FIG. 13 is a top view of the cover and cup implant with cross sectioned and detailed views, particularly FIG. 13A illustrating the cross sectioned view during assembly as surplus water is expelled and FIG. 13B illustrating the cross sectioned view fully assembled with a hydraulic seal;

FIG. 14 shows the cup with introducer being press fitted into the acetabulum (hip socket);

FIG. 15 shows the cup and cover fitted into the acetabulum;

FIG. 16 shows the cover being removed from the cup;

FIG. 17 is a cross sectioned view showing the cover being removed from the cup;

FIG. 18 is an isometric view of the offset introducer shaft;

FIG. 19 is a semi-exploded isometric view of the offset introducer shaft;

FIG. 20 is a side view with cross-sectioned and detailed views of the assembled cup implant, cover, and introducer shaft in the latched (locked) position;

FIG. 21 a cross sectioned/ detailed view of the assembled cup, cover, and introducer shaft in the unlatched (release) position;

FIG. 22 is an isometric view of the straight introducer shaft; and

FIG. 23 is a side view of the straight introducer shaft with cross-sectioned and detailed views.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE INVENTION

The present disclosure provides a cup holding solution which can be applied to any hip cup with large diameter bearing surface without the need for special holding features on the cup. It functions by establishing a hydraulic seal between a cover assembly and the cup implant which holds them together securely. The cover is connected via a separate mechanical connection to an introducer shaft which is used to position and forcibly impact the cup implant into the prepared acetabulum. Once fitted, the introducer is disconnected, leaving the cover still attached to the cup implant. The hydraulic seal between cover and cup implant is then broken by pulling a release tab on the cover enabling easy removal of the cover.

In United Kingdom patent document no. GB2552173A, a similar hydraulic seal is established between three elements, (1) the cup implant, (2) the cover and (3) the introducer, whereas on the latest invention it is established between only two elements, (1) the cup implant and (2) the cover. This provides several advantages, firstly, during implantation, the surgeon can remove and reconnect the Introducer from the cover without disrupting the hydraulic seal. This allows for checking of cup position in the acetabulum with the option of reattaching the Introducer to further advance or adjust cup position. Secondly, it is advantageous to be able to turn the cup implant when approaching the acetabulum to position asymmetric features on the cup rim to the intended positions to match the anatomy of the acetabular rim. Where a straight introducer shaft is employed, this can be done by rotating the entire assembly about the shaft’s axis, however where an offset shaft is employed it is desirable to turn the cup independently without influencing the shaft offset position, because, due to the constraints of surgical access, the offset shaft is positioned within a narrow window of available space within the surgical site. Furthermore, the optimum position for the offset varies depending on surgical approach and surgeon preference, so it is advantageous to position the offset shaft and cup rim rotational features independently of one another. It is easier to allow the cup with cover to turn independently of the introducer if the hydraulic seal does not rely on features on the introducer. A mechanism for rotating the cup via the introducer will be described later. Thirdly, because the hydraulic seal is contained within two elements, the required fluid volume is reduced, as is the likelihood of air bubbles remaining in the fluid system which reduce the holding force. And lastly, the non-return valve and releasing mechanism present on the previous invention are not necessary with the new invention so there and fewer components contributing to the hydraulic seal and less to go wrong.

The above published patent application describes a means for establishing low pressure in the fluid (or maintaining it under suction). Experience has shown that this is not essential, because a sufficient holding force is achieved by allowing excess fluid to escape from the sealed cavity and preventing air or fluid from re-entering. For this reason, the latest invention does not include description of a similar means to lower pressure. Furthermore, the above publication states that while it is desirable to use fluid to establish the holding force, it also functions without fluid, where only air is present in the sealed cavity, but that the holding force is reduced. It is also the case with the latest invention that it functions without fluid but with reduced holding force.

In this document emphasis is placed to the hydraulic seal between cup implant and cover. A description of the third element, the introducer is included for context. The cover and introducer may be supplied as sterile single use devices. However, alternatively they may be supplied as re-useable devices providing, they are decontaminated, cleaned, and sterilized before each use.

The main function is to establish a holding force between cup implant and cover which can be released once the cup implant is positioned correctly and without undermining the fit of the cup in the acetabular socket. The mating surfaces of cup implant and cover must be in full contact with one another to transfer the impaction force from the Introducer to the cup implant while being fitted into a slightly undersized acetabular socket by impacting the introducer with a surgical hammer. At the same time, the cup implant and in particular the super-finished bearing surface must be protected.

The cover includes a seal component (such as silicone rubber) and a cover body. In some arrangements, including preferable configurations, a connector (e.g. in the form of a snap-ring) can also be utilized. Interactions between the cover and cup implant are responsible for, (a) allowing excess fluid to escape enabling full contact between mating surfaces (b) establishing and maintaining a hydraulic seal, holding them together and (c) allowing the hydraulic seal to be disrupted, for removal of the cover once the cup is implanted correctly.

In use, the cup implant is filled with a fluid (e.g. sterile water) and the cover is rotationally aligned and pressed together with the filled cup implant. Either the cover and cup are pressed together separately, or the cover is first fitted to the introducer before being pressed onto the cup implant. As they are pressed together excess fluid is expelled around a portion of the seal until the mating surfaces contact one another. If assembled separately, the cup implant with cover are then fitted onto the introducer. The cup implant is then transferred to the surgical site via the introducer and aligned correctly prior to impaction into the acetabular socket by hammering the end of the introducer. During cup fitting, the introducer can be removed and reconnected from the cup with cover for inspection and assessment of position by the surgeon whilst the hydraulic seal remains intact. Once positioned correctly, the cover is removed from the cup by gripping and pulling one of the seal tabs protruding upwards from the cup rim, either using a finger grip or with the aid of forceps. Only light pulling force is required as will be described below.

To maintain the hydraulic seal a number of portions (typically three, although two, four, five, or more can also be utilized) of the seal are being compressed between the cover body and cup bearing surface including the areas adjacent to the tabs and one other. Elsewhere there is slight clearance for the seal between the cover body and cup bearing surface. This allows excess fluid to be expelled on assembly and in this region the hydraulic seal is maintained by the edges of the seal pressing against the bearing surface. Thus, a hydraulic seal is maintained all round either by compression of the seal or by its edges being in contact. The edges of the seal are biased towards contacting the bearing surface because the sealing (e.g. silicone) part, typically being cut from a flat sheet, is being deformed into a spherical shape whilst trying to return to its free shape through its resilient nature. This causes not only the seal edges, but also the outer seal surfaces adjacent to edges to be in contact with the bearing surface forming an effective fluid tight or airtight seal.

The seal is disrupted by pulling the tabs because, as the silicone stretches, it becomes thinner beyond the amount it is being compressed, opening up a small gap for air to enter the sealed cavity and disrupting the seal. It takes only light force to stretch the tabs and they can be stretched inwards (towards the center of the cup) therefore avoiding forces which tend to pull against the cup fixation.

In addition to the outer edges of the seal interacting with the cup, the inner edges must also form a seal with the cover body to make a fully sealed cavity. This can be achieved by sandwiching the inner seal edge between the cover body and the connector where present (e.g. snap-ring), compressing the silicone, and forming an effective secondary seal. Other ways of forming a secondary seal are well known to the skilled person and can include e.g. the seal element being bonded to the cover body (e.g. via a heat bond, adhesive bond, etc.) or by over-moulding the seal to the cover. Alternatively, an effective seal can be made by stretching the smaller diameter central hole of the seal into a groove of larger diameter on the cover, similar to stretching an o-ring into an o-ring groove on a shaft.

The cover also incorporates features to enable secure fitting of the introducer via a latching mechanism and a rotational drive feature so that the cup with cover can be rotated around the introducer axis. This is beneficial to align the cup rim features as intended to the anatomical features of the acetabular rim without fully rotating the introducer. The springloaded latch engages automatically as the introducer is assembled into the cover, it can then be rotated via an adjustment knob and locked in a certain rotational position. To disengage the introducer from the cup with cover, the knob is pulled back towards the operator which unlatches it.

Embodiments of covers for medical implants as well as systems, introducers and methods for their use are provided as follows:

A cover for a medical implant, the cover comprising a body portion and a seal, the body portion comprising a distal end and a proximal end, the proximal end comprising a region adapted for connecting to an introducer and the seal comprising at least one tab extending away in a proximal direction from the body portion. The at least one tab can be manipulated to deform the seal. The body portion may include at least one raised portion between the proximal and distal ends that assists in maintaining contact between the cover and the medical implant when in use. In some cases, the at least one raised portion is two raised portions. In some arrangements, there is at least one further raised portion approximately equidistant from the at least one raised portion. In some configurations the proximal end of the body portion comprises at least one slot configured to allow the at least one tab to pass through a respective slot. In some cases, there is a raised portion adjacent each of the at least one slot of the body portion. In some configurations, the seal extends over the at least one raised portion. The seal can comprise a plurality of, such as but not limited to two, tabs. The seal may be removably attachable to the body portion. The seal may be fixedly attached to the body portion at a region of fixed attachment, and wherein said region of fixed attachment forms an air- and/or watertight, or a substantially air- and/or watertight, seal between the seal and the body portion.

The body portion may be dimensioned in a region away from the region of fixed attachment to provide clearance between it and the seal, thereby allowing the seal to move towards and away from the longitudinal axis of the cover. In some arrangements the cover further comprises a connector which connects the seal to the body portion. The distal end of the body portion can be profiled at a connector region to accept the connector. The connector region can be profiled to retain the connector, optionally wherein the connector comprises projections which interact with a rim on the connector region. In some cases, the medical implant is a cup implant, and the cover is profiled to fit within the cup. The body portion comprises a rim, optionally wherein the rim is contoured, and optionally wherein the rim is contoured to match a rim contour of the medical implant. In some arrangements the seal is contoured to match a contour of a rim of the body portion. In some arrangements the rim of the seal sits distally from a rim of the body portion except for at the at least one tab.

Also provided is a method of fixing a cover to a medical implant, the method comprising: (i) optionally (e.g. if a liquid is being used instead of air) adding a fluid to the region of the medical implant which will accept the cover; (ii) introducing the cover into the region of the medical implant such that the cover displaces the fluid, until the cover abuts the surface of the medical implant; and (iii) optionally adding additional force to the cover such that the seal is compressed between the body portion and the medical implant. The method can further comprise releasing the cover from the medical implant, the additional steps comprising: (iv) manipulating the one or more tabs on the seal in order to disrupt the seal between the cover and the medical implant; and (v) removing the cover from the medical implant.

There is also provided a system comprising a cover for a medical implant and an introducer, wherein the cover is releasably fixable to the introducer, the introducer comprising a drive shaft and a connector hub, the introducer comprising latching means to secure the connector hub to the cover, optionally wherein the connector hub comprises a rim that engages with a complementary valley in a cover for a medical implant.

This is also provided a system comprising a medical implant and a cover, wherein the cover is releasably fixable to an inner surface of the medical implant.

Also disclosed herein is an introducer comprising a drive shaft and a connector hub, the introducer comprising latching means to secure the connector hub to a cover for a medical implant. The connector hub may comprise a rim that engages with a complementary valley in a cover for a medical implant. In some cases, the latching means is moveable with respect to the drive shaft. The drive shaft may be offset from a longitudinal axis of the connector hub. In such arrangements, the introducer comprises an impact shaft for transferring load from a handle region of the introducer to the connector hub, the impact shaft being distinct from the drive shaft. In some cases, the drive shaft is independently moveable with respect to the impact shaft such that the drive shaft can be rotated and/or moved towards and away from the connector hub (in some cases parallel to the longitudinal axis of the drive shaft).

In some arrangements the introducer comprises a means to bias the drive shaft towards the connector hub and, in some cases, the bias comprises a spring. The means to bias the drive shaft towards the connector hub may be independent from the latching means. The drive shaft has a distal end (i.e. the end at the connector hub) which may comprise an adaptor which allows the drive shaft to engage with and rotate a cover for a medical implant while at an offset angle, such as a ball drive end optionally with square sides. The drive shaft may be parallel to the longitudinal axis of the connector hub. The drive shaft of the introducer can act as an impact shaft for transferring load from a handle region of the introducer to the connector hub. In some arrangements the latching means is biased towards the connector hub, optionally wherein said bias is only present when the latching means is moved away from said connector hub. Some configurations have the connector hub comprising an anti-rotation feature, such as a projection. In some cases, the projection interacts with a complementary recess in the cover for the medical implant.

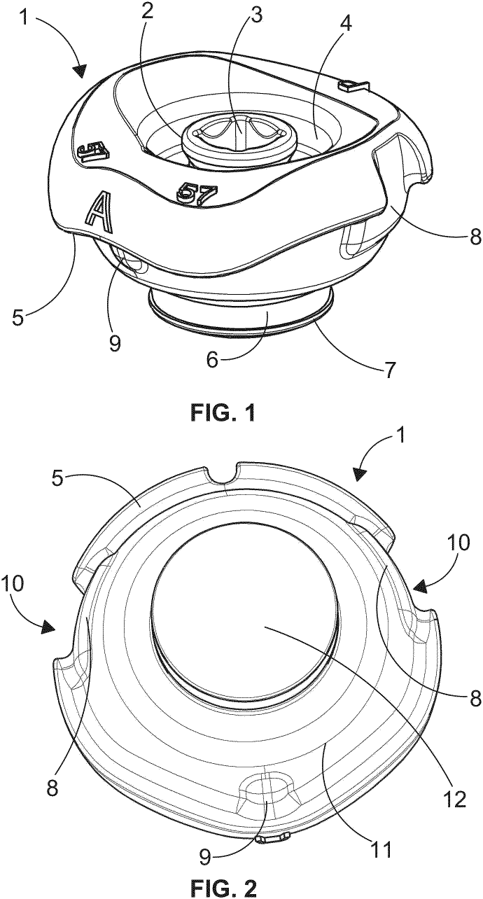

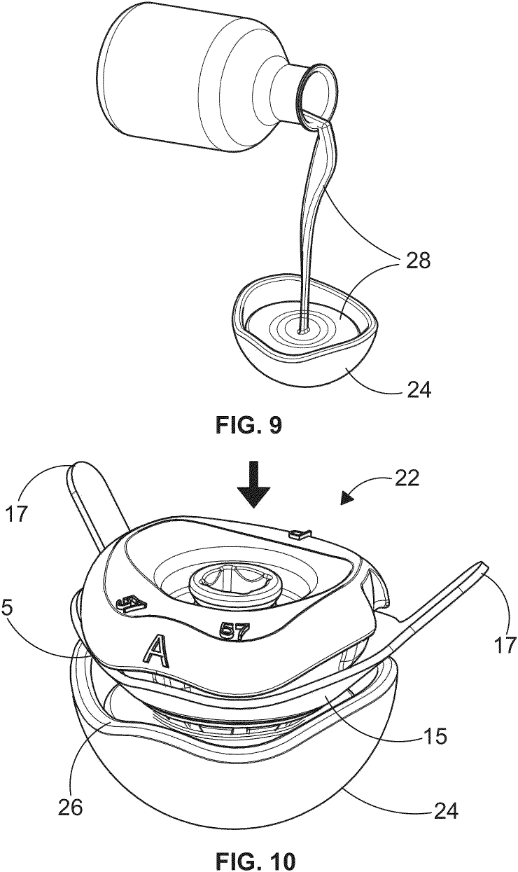

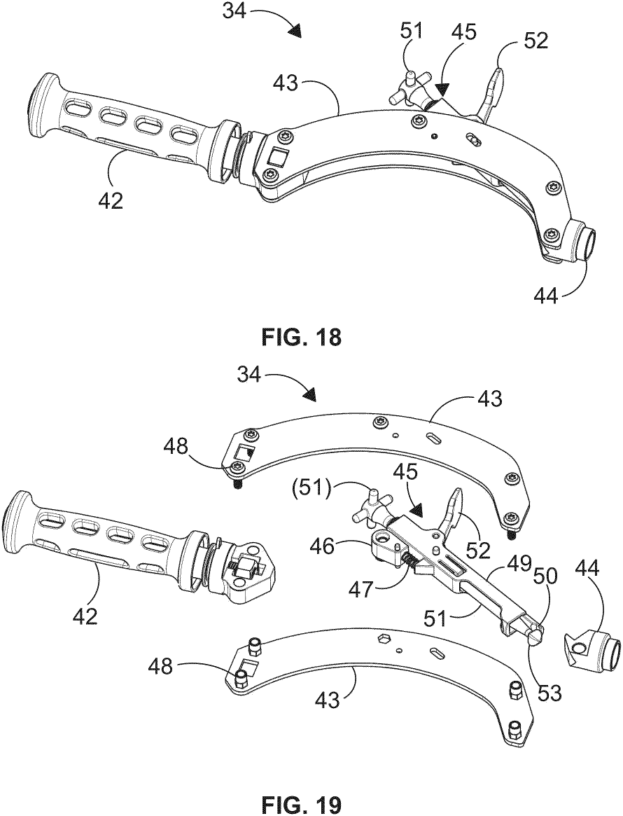

FIGS. 1 - 3 show the cover body [1] prior to assembly. The cover body can be manufactured from a suitable material to avoid damaging the cup implant during impaction, typically a polymeric material. The cover rim [5] can be profiled to match the contoured shape of the cup implant rim [26] (e.g. as shown in FIG. 8). Alternatively, the cover rim does not need to be profiled to match the cup implant rim. Recess [4] accepts the introducer hub [44 or 58] shown in FIGS. 18-23. Central boss [2] can optionally comprise a rim [13] and/or a recessed groove [14] on its outside diameter to accept introducer latch [50 or 62] shown in FIGS. 19-23. Central boss [2] can also optionally incorporate a square recess [3] which accepts engaging ball drive end [53] on the introducer [34]. The square recess alternatively could be hexagonal, or star shaped or any shape which can be driven by an off-axis drive shaft. The cap rim [5] may be interrupted by one or more slots [10] which accommodate tabs [17] on seal [15]. The cap has a generally spherical or spherical diameter which approximately matches the spherical bore of the cup implant [25] in FIG. 8. This spherical diameter has recessed region [11] and end region [12]. Recessed region [11] optionally has one or more, optionally two, raised pads [8] adjacent to slots [10] and optionally at least one additional raised pad [9] mid-way between slots [10]. These pads correspond to regions where the seal is compressed as will be described later. Cylindrical diameter [6] accepts snap-ring [19] which is retained by raised flange [7]. In some arrangements the cover will comprise two raised pads (i.e. one each adjacent to slots [10]) and an additional (third) raised pad between the two.

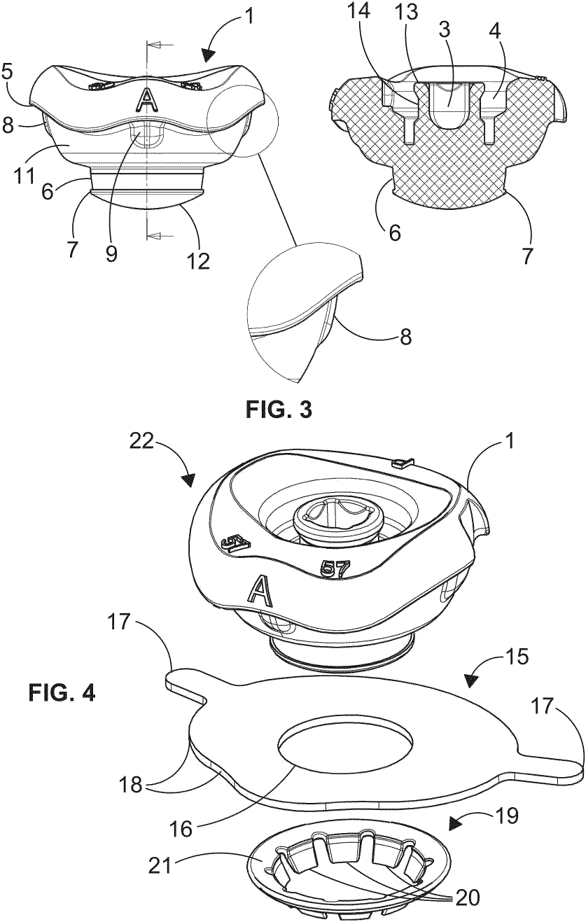

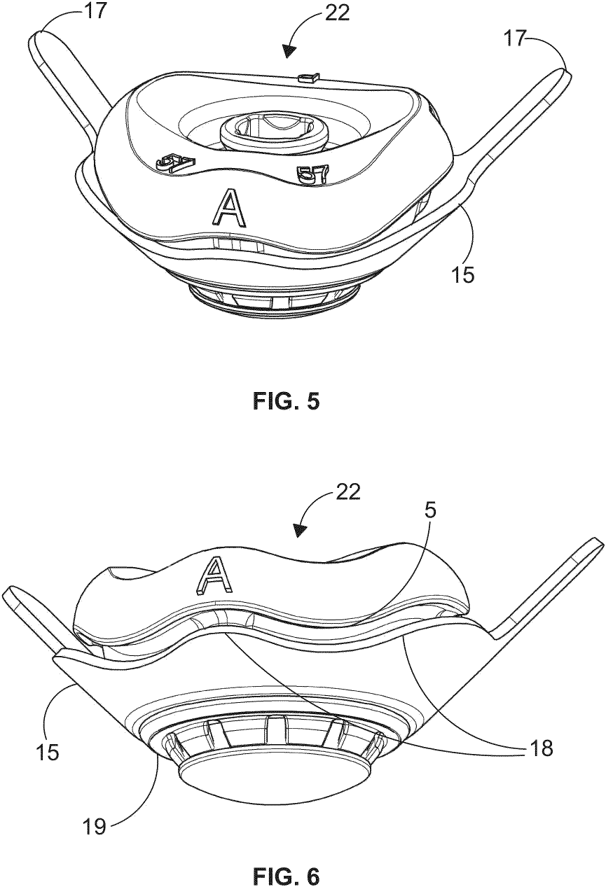

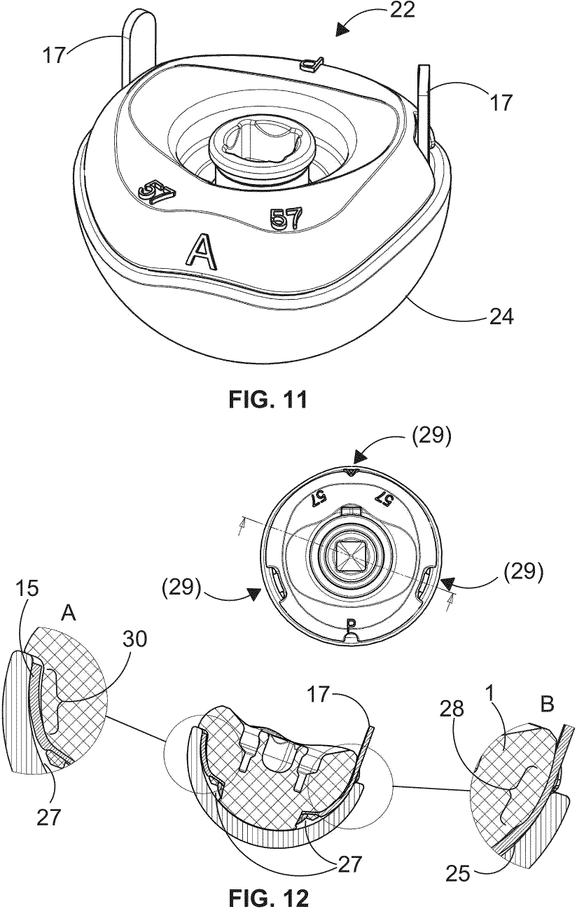

FIG. 4 is an exploded view showing an arrangement with all three component parts of cover [22], cover body [1], seal [15] and snap-ring [19]. As described above, the snap-ring can be optional and discussion herein of various elements in the context of arrangements of the cover with the snap-ring are equally applicable to arrangements not comprising the snap-ring. The seal is manufactured from a flexible elastomeric material such as silicone rubber, typically die-cut from flat sheet material that can be e.g. approximately 1.5 mm thick. Alternatively, the seal could be manufacture in nitrile rubber, thermoplastic elastomer (TPE), polyurethane or any other elastomeric material suitable for medical use of which the skilled person will be aware. The seal has circular central hole [16] which is smaller than cylindrical diameter [6] on cover body, such that, when it is stretched over on assembly, it deforms the flat seal into a conical shape (as shown in FIGS. 5-6). The outer profile [18] is optionally contoured such that when the cover is pressed together with the cup implant, the edges of the seal approximately match but remain just short of the cover rim [5] and the two tabs [17] protrude through slots [10] and beyond the cup rim as shown in FIGS. 11-15. The snap-ring [19], where present, is circular in shape with flange [21] and a multitude of fingers [20] around its inner circumference. The snap ring is manufactured form a similar polymeric material as the cover body such that the fingers are semi-flexible. Both the cover body and snap-ring can be 3d printed or moulded or machined in a polymeric material such as nylon.

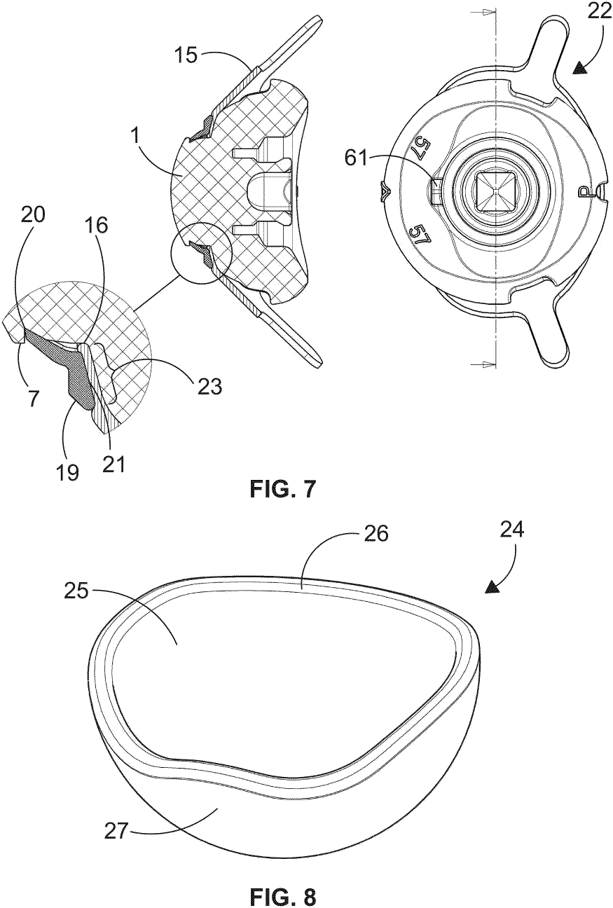

FIGS. 5-7 show the assembled cover [22] as it would be presented for use. FIG. 7 (cross sectioned and detailed view) shows seal region [23] adjacent to central hole [16] sandwiched between cover body [1] and flange [21] on snap-ring [19] and in region [23] the elastomeric material remains in a compressed state, forming a fluid tight/ airtight seal between seal [15] and cover body [1]. Also shown are the positions of the snap ring fingers [20] engaged with the cover body raised edge [7] retaining the snap ring in position and maintaining compression of the seal in region [23].

FIG. 8 shows a cup implant, typically used in HRA where the cup [24] is a hemispherical shell, consisting of inner surface [25] forming the bearing surface which is exceptionally smooth and accurately formed to match with a femoral head implant (not shown). The outer surface [27] typically has textured biocompatible surface and/or coating to be press-fitted directly into the patient’s acetabular socket. And rim 26 which is contoured in this embodiment but in alternative embodiments may also be flat/ planar. Typically, cup implants of this type are manufactured from zirconia toughened alumina ceramic (ZTA) or cross linked ultra-high molecular weight polyethylene (UHMWPE) or Cobalt Chrome Molybdenum alloy. In other embodiments (not shown) the cup implant may consist of outer shell and inner bearing preassembled together using a combination of the materials listed above.

In use, the cup implant [24] typically is removed from sterile packaging and placed on a flat surface. It is then filled with fluid (e.g. sterile water) [28] as shown in FIG. 9. With the cup resting on a flat surface to avoid spillage, the cover [22] is inserted into the cup [24] with contoured rims [5 & 26] (if present) approximately aligned as shown in FIG. 10. As they are pressed together excess fluid is expelled from a portion of the seal until the mating surfaces (cover rim [5] and cup rim [26]) are into contact with one another. FIG. 11 shows the cup with cover fully pressed together such that the mating surfaces are in full contact and being held together by the hydraulic seal. Alternatively, the cover is first fitted to the introducer before being pressed onto the cup implant. If assembled separately, the cup implant with cover are then fitted onto the introducer.

FIG. 12 includes a cross section of the cup and cover being held together by the hydraulic seal. As already described, to maintain sealed cavity [27] some portions of the seal are being compressed between the cover body and cup bearing surface including the areas adjacent to tabs [17]. Elsewhere there is slight clearance for the seal between the cover body and cup bearing surface. Due to this slight clearance the seal can fluctuate between an open position to expel excess fluid and closed position to maintain the sealed cavity.

FIG. 12B shows a portion [28] of seal [15] adjacent to the pull-tabs [17] being compressed to less than its free thickness between cover body [1] and cup bearing [25]. Where the cover comprises three raised pads as exemplified in the figure, compression occurs in three regions as indicated by arrows [29] adjacent to the two pull tabs [17] and a third location mid-way between the pull tabs. These areas of compression correspond to the three raised pads [8 & 9] on the cover body [1]. The third location [9] is included for stabilizing effect, ensuring compression is maintained particularly at the pull tab locations [8].

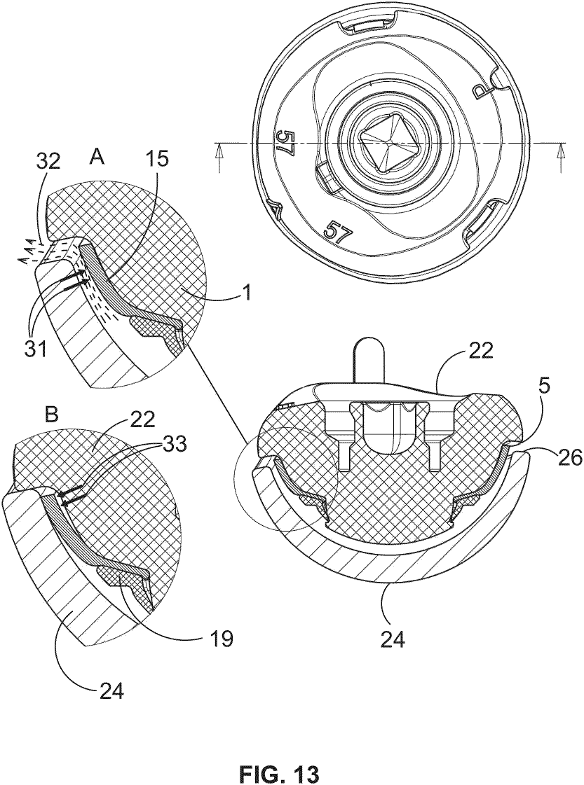

FIG. 12A shows a portion [30] where there is slight clearance for the seal [15] between the cover body [1] and cup bearing surface [25]. The seal occupies a gap slightly wider than the thickness of the seal itself, which allows excess fluid to be expelled on assembly around the outside of the seal which then closes up to prevent fluid or air from re-entering the sealed cavity. Those familiar with the field of fluid mechanics will appreciate that the seal acts in a similar way to an ‘umbrella check valve’ which allows fluid flow in one direction but prevents backflow in the opposite direction. In this embodiment excess fluid from the filled cup implant is allowed to escape because the pressure from the surplus fluid presses the seal towards the inside cover surface and away from the cup bearing surface, forming a slight opening. Once the excess fluid is expelled pressure normalizes and the seal regains contact with the cup bearing surface sealing the cavity. The edges of the seal are biased towards contacting the bearing surface due to the elastomeric bias in the material trying to return to its free form. This causes not only the seal edges, but also the outer seal surfaces close to the edges to be in flush contact with the bearing surface. In addition to this, if forces acting to separate the cover from the cup implant are present, for example as the cup is manipulated into the acetabulum, positive pressure on the inside surface of the seal presses the outer surface in flush contact with the bearing surface, preserving the seal and resisting separation. This is simulated in FIG. 13, where in 13A the seal [15] is pressed inwards towards the cover body [1] in direction of arrows [31] due to the pressure of surplus fluid escaping [32]. In 13B the seal is pressed outwards in direction of arrow [33] by (a) the elastomeric bias in the material and (b) positive pressure on the inside surface if forces tending to separate the cover from the cup are present.

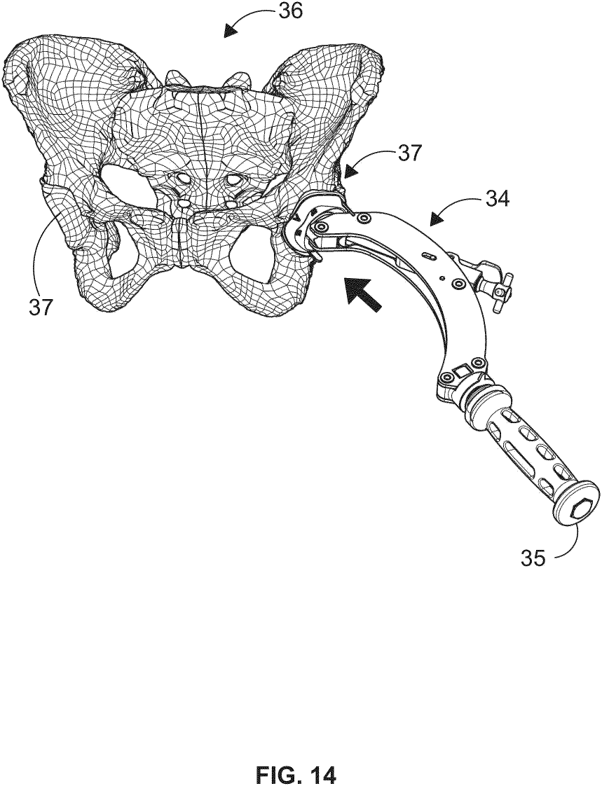

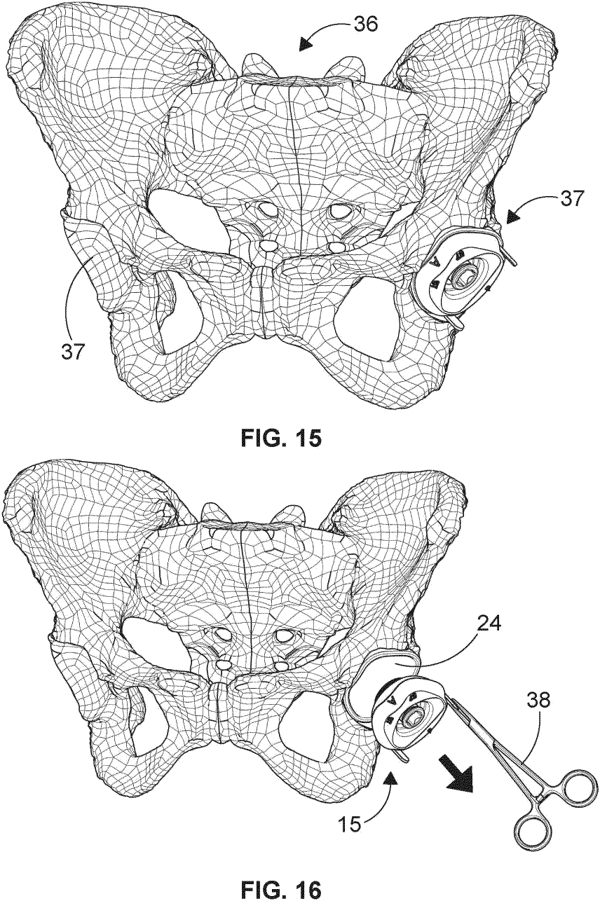

If assembled separately, the cup implant with cover are then fitted onto the introducer. With the hydraulic seal established, the cup implant can be transferred to the surgical site and manipulated into the pre-prepared acetabular socket [37] of the pelvis [36] in as shown in FIG. 14. This is done by impacting the distal end [35] of introducer [34] with a surgical hammer (not shown) until the cup is fully seated. As previously described the introducer [34] can be disconnected and reconnected to the cup with cover at any stage of impaction for inspection by the surgeon and/or upon final seating as shown in FIG. 15. Depending on the surgical approach, at least one of the tabs [17] is accessible for removing the cover by either gripping it with a finger grip, or with the aid of a standard surgical instrument such as forceps [38] as shown in FIG. 16. The seal is disrupted by pulling tabs [17] because, as the elastomeric material stretches, it becomes thinner beyond the amount it is being compressed in regions [28], opening up a small gap for air to enter the sealed cavity and disrupting the seal. It takes only light force to pull and stretch the tabs due to the relatively soft and stretchy nature of the elastomeric material. Furthermore, the tabs can be stretched inwards (towards the center of the cup) therefore avoiding forces which tend to pull against cup fixation.

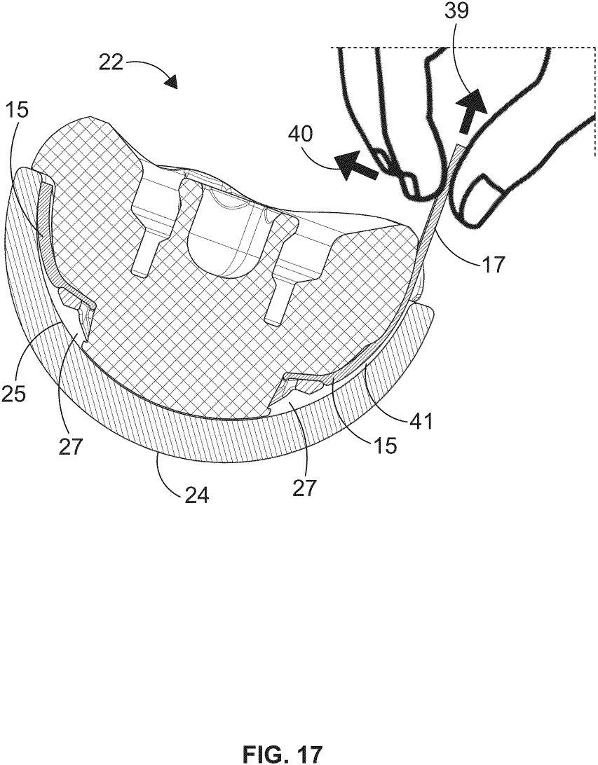

A simulation of the effect of stretching one of the tabs is shown in FIG. 17, where the tab [17] is being gripped between thumb and forefinger and pulled in the direction of arrow [39]. As the tab [17] is stretched, it becomes thinner until a small gap [41] opens up between the seal [15] and the cup bearing surface [25]. This allows air to enter the sealed cavity [27] and disrupts the hydraulic seal, allowing the cover to be easily removed. Alternatively, the tab can be pulled in direction of arrow [40] towards the center of the cup, thus avoiding any forces tending to pull the cup from the socket.

The embodiment of the introducer [34] shown in FIGS. 14 and 18-21 features an offset shaft which is favored by many surgeons because there are sometimes obstructions preventing direct access to the acetabulum with a straight introducer shaft, particularly with anterior surgical approaches and/or where surgical access is minimized to reduce incision size. It consists of handle [42], offset shaft [43], connector hub [44], latch sub-assembly [45] spring holder [46] and spring [47]. The offset shaft in this arrangement is made up of two plates, and the entire assembly is fastened together by a multitude of rivet nuts [48]. However, it will be appreciated that the offset shaft can be made in a single piece or in multiple pieces, and the means to fasten the assembly together is not limited to rivet nuts but can be any appropriate fastening means known to the skilled person.

The latch sub-assembly [45] incorporates housing [49] which incorporates latch [50] for retaining the cover, a rotating drive shaft [51] for rotating of the cover about the long axis of the introducer and a latching means such as a cam lever [52] to lock rotating drive shaft in a certain position. The drive shaft has an adaptor at its distal end to allow rotation of the cover whilst the drive shaft is off set. In the depicted arrangement this adaptor is shown as a ball drive end [53] with square sides which engages into recessed square drive feature [3] on the cover. The ball drive end [53] is similar to a standard ball ended Allen key which allows the tool to be used at an angle off-axis to the screw, except that the Allen key ball drive has hexagonal sides and this has square sides. In this case, the ball drive end allows the rotation drive shaft to be off axis relative to the axis of cover rotation. This is necessary due to the offset shape of the introducer shaft. Alternatively, the ball drive end [53] and mating recess [3] on cover body [1] could be hexagonal, or star shaped or any shape which allows rotation via and off-axis drive shaft.

Latch sub-assembly [45] is free to slide a short distance in the direction parallel to the drive shaft axis, while the position of spring holder [46] is fixed (e.g. by a rivet nut) so that the spring [47] biases the Latch sub-assembly towards hub [44]. This allows latch [50] and ball drive [53] to engage and disengage from the cover. When the cover is assembled together with the introducer, hub [44] engages into cover recess [4]. The latch and ball drive are driven back in direction of arrows [54] by cover boss rim [13] until the latch and ball drive drop into their respective engagements. This occurs automatically under the spring force as the operator pushes them together. To disengage and release the cover the operator must first pull the latch sub-assembly via the knob on drive shaft [51] a distance (e.g. 3-4 millimeters) against the spring force in direction of arrow [54] in FIG. 21. The latching means (e.g. cam lever [52]) to lock drive shaft rotation is independent of the operation of the latch sub-assembly and can be engaged or disengaged at any time.

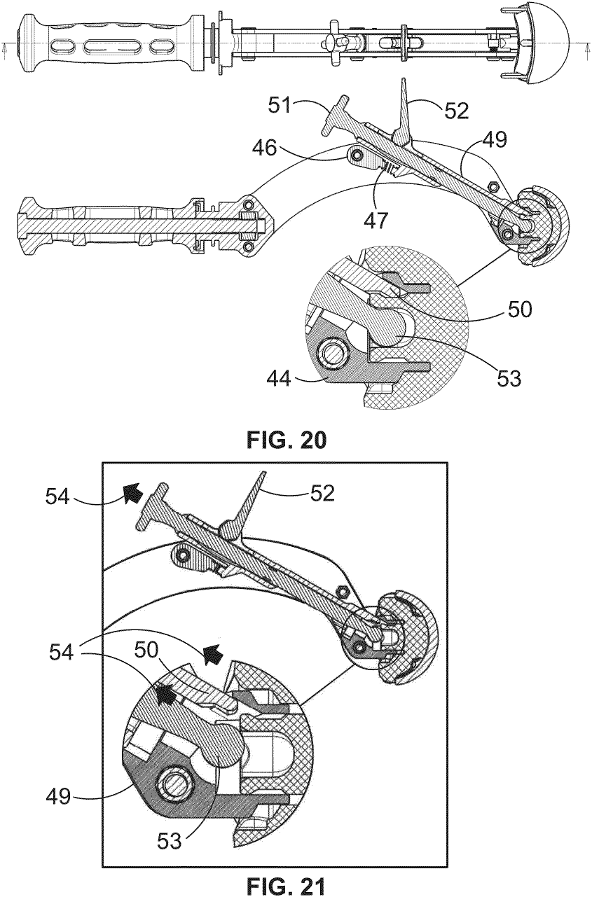

FIG. 20 shows the latch and ball drive in the engaged position and FIG. 21 shows the latch and ball drive in the disengaged position with latch assembly pulled back in direction of arrows [54].

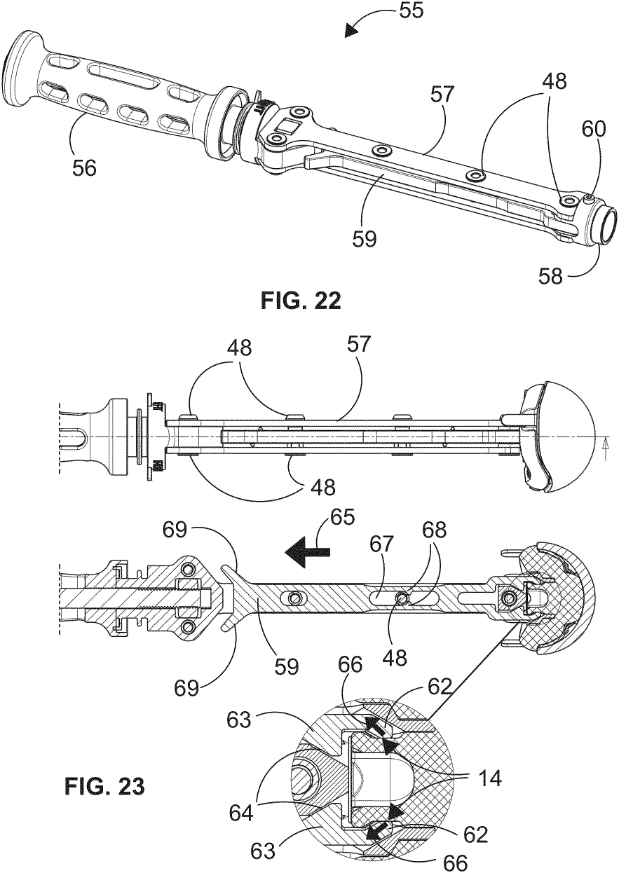

An alternative configuration of the introducer with a straight shaft [55] is shown in FIGS. 22 & 23. The straight introducer includes handle [56], shaft [57], connector hub [58] and carriage [59] secured together (e.g. by a multitude of rivet nuts [48]). In the case of a straight shaft the mechanism for rotating the cover is not necessary to be included because the entire introducer can be turned to orientate the cup implant in the appropriate rotation. The straight introducer connector hub optionally incorporates an anti-rotation pin [60] which engages in a complementary slot [61] in cover body [1] so that the cover and introducer rotate in unison (the offset introducer does not need to include such a pin, although it can be present in some arrangements). The straight introducer includes carriage [59] which is free to slide in the direction parallel to shaft axis. The carriage incorporates latches [62] (optionally two) which engage with recess in cover boss [14]. The latches are on semi-flexible fingers/prongs [63] which engage with a portion [64] (optionally triangular in shape) of hub [58]. As the carriage moves in direction of arrow [65], the latch prongs move along (triangular) portion [64] so the resultant movement is in direction of arrows [66] moving the latches out of cover boss recess [14] to release the cover. The skilled person will be aware that other suitable geometries can be used to achieve a similar effect of forcing the latch fingers outwards as the carriage is moved away from the connector hub. In place of a spring, the carriage includes slot [67] with constrictions [68] (in this arrangement two constrictions are shown) located adjacent to one of three rivet nuts [48]. As the carriage moves back in direction of arrow [65], the constrictions [68] move up over the rivet nut (or other similar pin) diameter with the effect of biasing the carriage towards hub [58] and acting like a spring. The skilled person will be aware of other biasing means that could be utilized to achieve a similar effect (e.g. use of a spring). The carriage also includes flanges [69] which form a finger grip to release the latch.

The cover is universal so that it will accommodate either straight or offset introducers depending on surgeon preference. The cover may be supplied sterile for single-use or could be cleaned, re-sterilized and re-used several times. Those skilled in the art will appreciate that it is advantageous to have a commonly designed cover to fit both straight and offset introducers to improve compatibility and reduce instrument inventory.

It will be appreciated to those skilled in the art having the benefit of this disclosure that this invention is believed to provide covers for medical implants as well as systems, introducers, and methods of their use. Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A cover for a medical implant, the cover comprising a body portion and a seal, the body portion comprising a distal end and a proximal end, the proximal end comprising a region adapted for connecting to an introducer and the seal comprising at least one tab extending away in a proximal direction from the body portion.

2. The cover according to claim 1, wherein the at least one tab can be manipulated to deform the seal.

3. The cover according to claim 1, wherein the body portion comprises at least one raised portion between the proximal and distal ends that assists in maintaining contact between the cover and the medical implant when in use.

4. The cover according to claim 3, wherein there is at least one further raised portion approximately equidistant from the at least one raised portion.

5. The cover according to claim 3, wherein the seal extends over the at least one raised portion.

6. The cover according to claim 1, wherein the proximal end of the body portion comprises at least one slot configured to allow the at least one tab to pass through a respective slot.

7. The cover according to claim 5, wherein there is a raised portion adjacent each of the at least one slot of the body portion.

8. The cover according to claim 1, wherein the medical implant is a cup implant, and the cover is profiled to fit within the cup.

9. A system comprising:
a medical implant; and
a cover releasably fixable to an inner surface of the medical implant, wherein the cover comprises a body portion and a seal, wherein the body portion comprises a distal end and a proximal end, wherein the proximal end comprises a region adapted for connecting to an introducer and, and wherein the seal comprises at least one tab extending away in a proximal direction from the body portion.

10. An introducer comprising a drive shaft, a connector hub, and a latching means to secure the connector hub to a cover for a medical implant.

11. The introducer according to claim 10, wherein the cover comprises a body portion and a seal, wherein the body portion comprises a distal end and a proximal end, wherein the proximal end comprises a region adapted for connecting to the introducer and, and wherein the seal comprises at least one tab extending away in a proximal direction from the body portion.

12. The introducer of claim 10, wherein the connector hub comprises a rim that engages with a complementary valley in a cover for a medical implant.

13. The introducer of claim 10, wherein the latching means is moveable with respect to the drive shaft.

14. The introducer of claim 10, wherein the drive shaft is offset from a longitudinal axis of the connector hub.

15. The introducer of claim 10, wherein the introducer comprises an impact shaft for transferring load from a handle region of the introducer to the connector hub, the impact shaft being distinct from the drive shaft.

16. The introducer of claim 15, wherein the drive shaft is independently moveable with respect to the impact shaft such that the drive shaft can be rotated and/or moved towards and away from the connector hub.

17. The introducer of claim 10, wherein the introducer comprises means to bias the drive shaft towards the connector hub.

18. The introducer of claim 17, wherein the means to bias the drive shaft towards the connector hub is independent from the latching means.

19. The introducer of claim 10, wherein the drive shaft has a distal end comprising an adaptor which allows the drive shaft to engage with and rotate a cover for a medical implant while at an offset angle.

20. The introducer of claim 10, wherein the drive shaft of the introducer acts as an impact shaft for transferring load from a handle region of the introducer to the connector hub.
